(19) United States
(12) Patent Application Publication
(10) Pub. No.: US 2012/0189860 A1
(43) Pub. Date: Jul. 26, 2012

(54) POLYMERIC COMPOSITIONS COMPRISING POLYLACTIC ACID OLIGOMERS AND METHODS OF MAKING THE SAME

(75) Inventors: Fengkui LI, Houston, TX (US); John ASHBAUGH, Houston, TX (US)

(73) Assignee: Fina Technology, Inc., Houston, TX (US)

(21) Appl. No.: 13/012,157

(22) Filed: Jan. 24, 2011

(51) Int. Cl. B32B 27/32; C07C 67/30; C08L 67/04; C08G 63/91

(52) U.S. Cl. 428/500; 525/437; 560/180; 525/440.04; 525/445; 525/186

(57) ABSTRACT

Process of modifying polylactic acid and compositions formed therefrom are described herein. The process generally includes providing a first polylactic acid, wherein the first polylactic acid includes a carboxylic acid end group and unsaturating the first polylactic acid to form a second polylactic acid.

### FIELD

Embodiments of the present invention generally relate to polymeric materials including biodegradable polymers. In particular, embodiments relate to polymeric materials including polylactic acid (PLA) oligomers.

### BACKGROUND

Synthetic polymeric materials, such as polypropylene and polyethylene resins, are widely used in the manufacturing of a variety of end-use articles ranging from medical devices to food containers. While articles constructed from synthetic polymeric materials have widespread utility, these materials tend to degrade slowly, if at all, in a natural environment. In response to environmental concerns, interest in the production and utility of more readily biodegradable polymeric materials has been increasing. These materials, also known as “green materials”, may undergo accelerated degradation in a natural environment. The utility of these biodegradable polymeric materials (e.g., biopolymers) is often limited by their poor mechanical and/or physical properties. Thus, a need exists for development of biodegradable polymeric compositions having desirable physical and/or mechanical properties.

In an effort to overcome poor mechanical properties, biopolymers have been blended with synthetic polymers by using conventional plastic processing tools. However, efforts continue to improve PLA polymer properties.

### SUMMARY

Embodiments of the present invention include a process of modifying polylactic acid. The process generally includes providing a first polylactic acid, wherein the first polylactic acid includes a carboxylic acid end group and unsaturating the first polylactic acid to form a second polylactic acid.

In one or more embodiments, the process of the preceding paragraph further includes contacting the first polylactic acid with a dual functional compound.

In one or more embodiments of the process of any preceding paragraph, the first polylactic acid is represented by the formula:

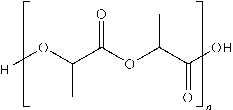

wherein n is a discrete number.

In one or more embodiments of the process of any preceding paragraph, the first polylactic acid is an oligomer.

In one or more embodiments of the process of any preceding paragraph, the first polylactic acid exhibits a number average molecular weight of from about 500 g/mol to about 200,000 g/mol.

In one or more embodiments of the process of any preceding paragraph, the first polylactic acid exhibits a number average molecular weight of from about 1000 g/mol to about 20,000 g/mol.

In one or more embodiments of the process of any preceding paragraph, the dual functional compound includes a first functional group comprises a carbon-carbon double bond on one end and a second functional group capable of reacting with the carboxylic acid end group.

In one or more embodiments of the process of any preceding paragraph, the second functional group is selected from epoxy groups, isocyanate groups and combinations thereof.

In one or more embodiments of the process of any preceding paragraph, the dual functional compound is selected from glycidyl methacrylates, unsaturated isocyanates, epoxidized butadiene and combinations thereof.

In one or more embodiments of the process of any preceding paragraph, the contact comprises reactive extrusion.

One or more embodiments include a modified polylactic acid of any preceding paragraph.

One or more embodiments include a process of forming polymeric compositions. The process generally includes providing an olefinic group and contacting the olefinic group with the modified polylactic acid of any preceding paragraph under polymerization conditions to form a polymeric composition.

In one or more embodiments of the process of any preceding paragraph, the olefinic group is selected from styrene, acrylate, and combinations thereof.

In one or more embodiments of the process of any preceding paragraph, the olefinic group is an unsaturated polyolefin comprising polybutadiene.

One or more embodiments include polymeric blends. The polymeric blends generally include a third polylactic acid, an olefin based polymer and a polymeric composition formed by the process of any preceding paragraph, wherein the polymeric composition is adapted to compatibilize the third polylactic acid and olefin based polymer.

One or more embodiments include a co-extruded article. The co-extruded article generally includes a first layer including a third polylactic acid, a second layer including an olefin based polymer and a tie layer including a polymeric composition formed by the process of any preceding paragraph.

In one or more embodiments of the process of any preceding paragraph, the contact occurs in the presence of peroxide.

One or more embodiments include a process of forming polymeric compositions include polymerizing an unsaturated polylactic acid formed by contacting a first polylactic acid with a dual functional compound to form an unsaturated polylactic acid, wherein the first polylactic acid is represented by the formula:

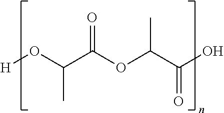

wherein n is a discrete number and polymerizing the unsaturated polylactic acid under polymerization conditions to form a polymeric composition.

### DETAILED DESCRIPTION

### Introduction and Definitions

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions when the information in this patent is combined with available information and technology.

Various terms as used herein are shown below. To the extent a term used in a claim is not defined below, it should be given the broadest definition skilled persons in the pertinent art have given that term as reflected in printed publications and issued patents at the time of filing. Further, unless otherwise specified, all compounds described herein may be substituted or unsubstituted and the listing of compounds includes derivatives thereof.

Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations.

Polylactic acid (PEA) polymer is a biodegradable, thermoplastic, aliphatic polyester derived from renewable resources, such as corn starch or sugarcane. Bacterial fermentation may be used to produce lactic acid from corn starch or cane sugar, for example. Lactic acid can be directly polymerized to a low molecular weight PEA, often referred to as PLA oligomer, and then catalytically dimerized to make the cyclic monomer called lactides. PEA oligomer often has poor thermal stability and inferior mechanical properties compared to high molecular weight PLA. High molecular weight PLA (e.g., Mn=20,000˜100,000) is typically produced by ring-opening polymerization of lactides. As used herein, both PLA oligomers and high molecular weight PLA will collectively be referred to as PLA. When referring specifically to either PEA oligomers or high molecular weight PLA, such will be referred to independently.

PLA may be selected from poly-L-lactide (PLEA), poly-D-lactide (MLA), poly-LD-lactide (PDLLA) and combinations thereof. PLA may be formed by known methods, such as dehydration condensation of lactic acid (see, U.S. Pat. No. 5,310,865, which is incorporated by reference herein) or synthesis of a cyclic lactide from lactic acid followed by ring opening polymerization of the cyclic lactide (see, U.S. Pat. No. 2,758,987, which is incorporated by reference herein), for example. Such processes may utilize catalysts for polylactic acid formation, such as tin compounds (e.g., tin octylate), titanium compounds (e.g., tetraisopropyl titanate), zirconium compounds (e.g., zirconium isopropoxide), antimony compounds (e.g., antimony trioxide) or combinations thereof, for example.

PLA may have a density of from about 1.105 glee to about 1.265 g/cc, or from about 1.205 glee to about 1.26 g/cc or from about 1.245 g/cc to about 1.255 g/cc (as determined in accordance with ASTM D792), for example.

PLA may have a number average molecular weight of from about 500 g/mol to about 100,000 g/mol, or from about 2000 g/mol to about 50,000 g/mol, or from about 4000 g/mol to about 30,000 g/mol, for example.

In one or more specific embodiments, PLA may include any PLA including a carboxylic acid end group, as illustrated below, wherein n is a discrete number (in contrast to a polymer wherein n may be unlimited), such as 2, 3 or 4, for example.

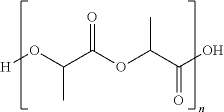

One or more embodiments include modification of PLA. The modification generally includes unsaturation of the backbone of the PLA. Such unsaturation may be introduced to the PLA through contact with dual functional compounds (i.e., bifunctional compounds), for example.

In one or more embodiments, the dual functional compounds may include compounds containing both a first functional group and a second functional group, wherein the first functional group comprises a carbon-carbon double bond on one end of the compound. The second functional group may include any functional group capable of reacting with the carboxylic acid end group of the PLA. In one or more embodiments, the second functional group is selected from epoxies, and isocyanate groups, for example. The dual functional compounds may be formed by known methods or obtained commercially, for example.

Examples of suitable dual functional compounds include dual functional monomers such as glycidyl methacrylate, which is commercially available from Dow Chemicals, and TMI® unsaturated isocyanate (i.e., TMI® (meta) unsaturated aliphatic isocyanate, which is commercially available from Cytec Industries, Inc.) and combinations thereof, for example.

In one specific example, unsaturated PLA may be formed by reactive extrusion of glycidyl methacrylate and PLA. The reaction of PLA oligomer and glycidyl methacrylate is illustrated below.

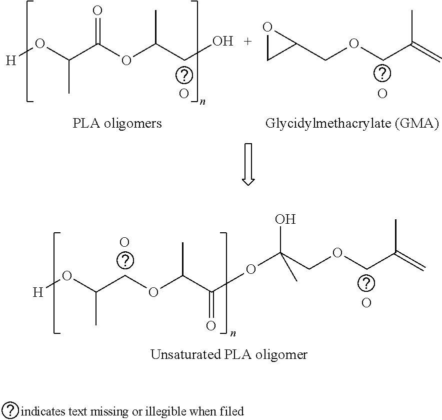

In another specific example, unsaturated PLA may be formed by reactive extrusion of TMI® (meta) unsaturated aliphatic isocyanate and PLA. The reaction of PLA oligomer and TMI® (meta) unsaturated aliphatic isocyanate is illustrated below.

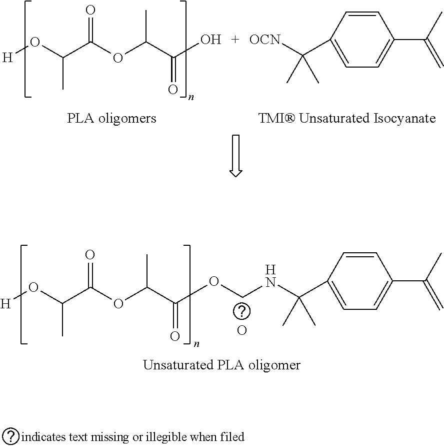

It is contemplated that in one or more embodiments, the unsaturated PLA may be directly polymerized with an optional unsaturated comonomer to form a polymeric composition. The term “unsaturated comonomer” refers to a comonomer having at least one double (or triple) carbon-carbon bond. Examples of suitable unsaturated comonomers generally include styrene, acrylates, unsaturated olefinic monomers, and combinations thereof, for example.

The polymerization may occur via free radical polymerization in the presence of an initiator peroxide, for example. Such embodiments are capable of producing products having structures that are comb-shaped (e.g., polymers having a main chain including a semi-rigid backbone and comb segments in the form of flexible and monodispersed side chains) or highly branched. As a result, these polymer products advantageously exhibit rheological behavior that is beneficial for improving melt processing of a wide variety of polymers, such as increased melt viscosity, for example.

In one or more embodiments, the unsaturated PLA can further copolymerize with unsaturated polyolefins to form polyolefin-PLA copolymers. One example of an unsaturated polyolefin is polybutadiene such as Krasol series, commercially available from Cray Valley, Inc. The reaction of unsaturated PLA oligomer with polybutadiene is illustrated below. The resulting polyolefin-PLA copolymers could be used as compatibilizers for hydrophobic polymer/hydrophilic polymer blends such as polyolefin (PO)/PLA or PO/polyethylene terephthalate (P. T) blends for example or tie layers for hydrophobic polymer/hydrophilic polymer blends such as PO/PLA or PO/PET blends for examples.

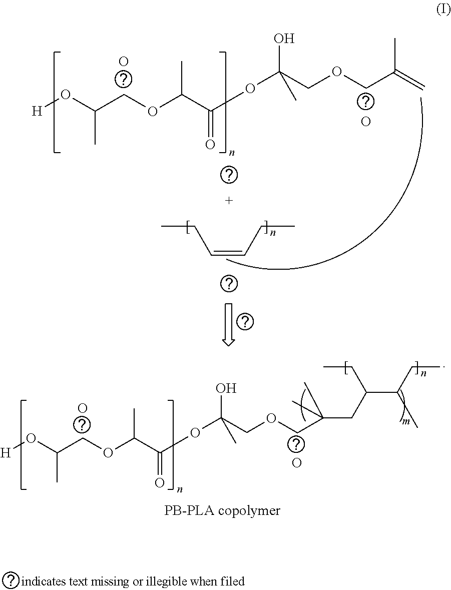

In one or more embodiments, the PLA can further copolymerize with specialty polyolefins to form polyolefin-PLA copolymers. One example of a specialty polyolefin is epoxidized polybutadiene, such as Poly bd® 600 and Poly bd® 605 commercially available from Cray Valley, Inc. The reaction of PLA oligomer with epoxidized polybutadiene is illustrated below. The resulting polyolefin-RLA copolymers could be used as compatibilizers for hydrophobic polymer/hydrophilic polymer blends, such as polyolefin (PO)/PLA or PO/polyethylene terephthalate (PET) blends, for example, or tie layers for hydrophobic polymer/hydrophilic polymer blends, such as PO/PLA or PO/PET blends for examples.

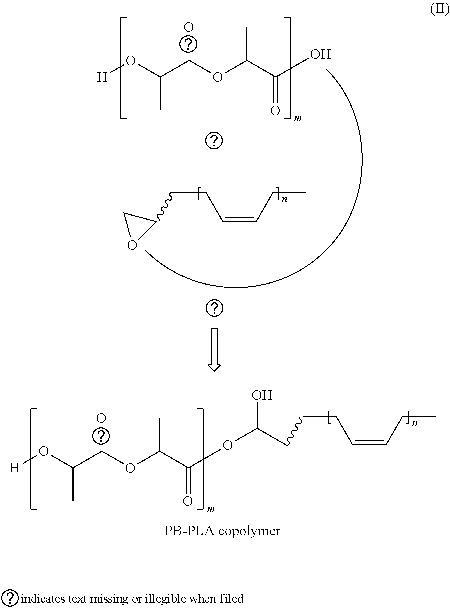

In one or more embodiments, the unsaturated PLA can further polymerize or copolymerize with other unsaturated groups when combined with PO, including polypropylene and polyethylene, and combinations thereof, with a radical source such as peroxide to form polyolefin/PLA in situ blends. The formation of polyolefin-PLA in situ blends is illustrated below.

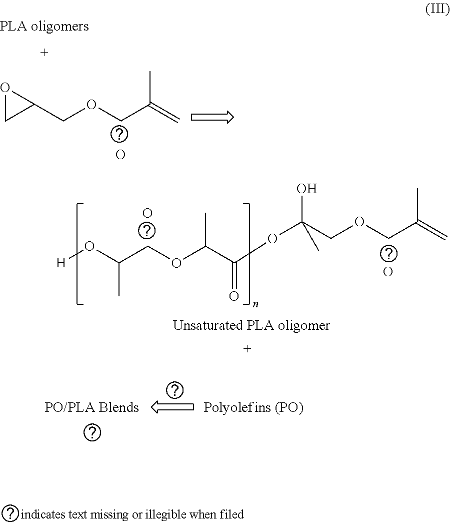

In one or more embodiments, the polymeric compositions are formed of an olefin based polymer, polylactic acid, and a polyolefin-PLA copolymer, such as those described above as compatibilizers.

In one or more embodiments, the polymeric compositions are formed of co-extruded olefin based polymer, polylactic acid, and a polyolefin-PLA copolymer, such as those described above as tie layers.

The polyolefin compositions may be formed by a variety of methods. For example, such formation may include blending of the olefin based polymer and the polylactic acid under conditions suitable for the formation of a blended material. Such blending may include dry blending, extrusion, mixing or combinations thereof, for example.

Alternatively, such formation may include utilizing a multi-layer structure to form the polymeric composition. The multi-layer structure may include a polyolefin layer and a PLA layer. The polyolefin layer and the PLA layer may be tied by a layer disposed between the polyolefin layer and the PLA layer (i.e., a tie layer). The tie layer may be formed of the polyolefin-PLA copolymers, for example.

In one or more embodiments, the olefin based polymer is selected from polypropylene, polyethylene, and combinations thereof.

In one or more embodiments, the olefin based polymers include propylene based polymers. As used herein, the term “propylene based” is used interchangeably with the terms “propylene polymer” or “polypropylene” and refers to a polymer having at least about 50 wt. %, or at least about 70 wt. %, or at least about 75 wt. %, or at least about 80 wt. %, or at least about 85 wt. % or at least about 90 wt. % polypropylene relative to the total weight of polymer, for example.

The propylene based polymers may have a molecular weight distribution (Mₙ/M_{w}) of from about 1.0 to about 20, or from about 1.5 to about 15 or from about 2 to about 12, for example.

The propylene based polymers may have a melting point (Tₘ) (as measured by DSC) of at least about 110° C., or from about 115° C. to about 175° C., for example.

The propylene based polymers may include about 15 wt. % or less, or about 12 wt. % or less 12, or about 10 wt. % or less, or about 6 wt. % or less, or about 5 wt. % or less or about 4 wt. % or less of xylene soluble material (XS), for example (as measured by ASTM D5492-06).

The propylene based polymers may have a melt flow rate (MFR) (as measured by ASTM D-1238) of from about 0.01 dg/min to about 2000 dg/min., or from about 0.01 dg/min. to about 100 dg/min., for example.

In one or more embodiments, the polymers include ethylene based polymers. As used herein, the term “ethylene based” is used interchangeably with the terms “ethylene polymer” or “polyethylene” and refers to a polymer having at least about 50 wt. %, or at least about 70 wt. %, or at least about 75 wt. %, or at least about 80 wt. %, or at least about 85 wt. % or at least about 90 wt. % polyethylene relative to the total weight of polymer, for example.

The ethylene based polymers may have a density (as measured by ASTM D-792) of from about 0.86 g/cc to about 0.98 g/cc, or from about 0.88 g/cc to about 0.965 g/cc, or from about 0.90 g/cc to about 0.965 g/cc or from about 0.925 g/cc to about 0.97 g/cc, for example.

The ethylene based polymers may have a melt index (MI₂) (as measured by ASTM D-1238) of from about 0.01 dg/min to about 100 dg/min., or from about 0.01 dg/min. to about 25 dg/min., or from about 0.03 dg/min. to about 15 dg/min. or from about 0.05 dg/min. to about 10 dg/min, for example.

In one or more embodiments, the olefin based polymers include low density polyethylene. In one or more embodiments, the olefin based polymers include linear low density polyethylene. In one or more embodiments, the olefin based polymers include medium density polyethylene. As used herein, the term “medium density polyethylene” refers to ethylene based polymers having a density of from about 0.92 g/cc to about 0.94 g/cc or from about 0.926 g/cc to about 0.94 g/cc, for example.

In one or more embodiments, the olefin based polymers include high density polyethylene. As used herein, the term “high density polyethylene” refers to ethylene based polymers having a density of from about 0.94 glee to about 0.97 glee, for example.

In an embodiment, the biodegradable polymeric composition, the olefin based polymer, the polylactic acid, the polyolefin-PLA copolymers or combinations thereof may contain additives to impart desired physical properties, such as printability, increased gloss, or a reduced blocking tendency. Examples of additives may include, without limitation, stabilizers, ultra-violet screening agents, oxidants, anti-oxidants, anti-static agents, ultraviolet light absorbents, lire retardants, processing oils, mold release agents, coloring agents, pigments/dyes, fillers or combinations thereof, for example. These additives may be included in amounts effective to impart desired properties.

The polymeric composition may exhibit a melt flow rate of from about 0.5 g/10 min. to about 500 g/10 min., or from about 1.5 g/10 min. to about 50 g/10 min. or from about 5.0 g/10 min. to about 20 g/10 min, for example. (MFR as defined herein refers to the quantity of a melted polymer resin that will flow through an orifice at a specified temperature and under a specified load. The MFR may be determined using a dead-weight piston Plastometer that extrudes polypropylene through an orifice of specified dimensions at a temperature of 230° C. and a load of 2.16 kg in accordance with ASTM D1238 condition L.)

The polymeric compositions are useful in applications known to one skilled in the art to be useful for conventional polymeric compositions, such as forming operations (e.g., film, sheet, pipe and fiber extrusion and co-extrusion as well as blow molding, injection molding and rotary molding). Films include blown, oriented or cast films formed by extrusion or co-extrusion or by lamination useful as shrink film, cling film, stretch film, sealing films, oriented films, snack packaging, heavy duty bags, grocery sacks, baked and frozen food packaging, medical packaging, industrial liners, and membranes, for example, in food-contact and non-food contact application. Fibers include slit-films, monofilaments, melt spinning, solution spinning and melt blown fiber operations for use in woven or non-woven form to make sacks, bags, rope, twine, carpet backing, carpet yarns, filters, diaper fabrics, medical garments and geotextiles, for example. Extruded articles include medical tubing, wire and cable coatings, sheets, such as thermoformed sheets (including profiles and plastic corrugated cardboard), geomembranes and pond liners, for example. Molded articles include single and multi-layered constructions in the form of bottles, tanks, large hollow articles, rigid food containers and toys, for example.

### EXAMPLES

### Example 1

Thermogravimetric analysis (TGA) was first used to characterize thermal stability of two PLA oligomers, in order to determine proper temperature range for melt modification of PLA oligomers. A commercial PLA polymer was thermally stable below 250° C. as shown in FIG. 1. NatureWorks recommended a melt processing temperature of less than 215° C. to minimize PLA degradation and retain PLA performances. In comparison, the two KA oligomers started to degrade at 100-120° C. Thus, to avoid significant degradation, all modifications of PLA oligomers were conducted at −100° C.

### Example 2

The modification of PLA oligomers with dual functional monomers,

GMA and TMI, was conducted at 100° C. in a Haake internal mixer. As the viscosities were very low, process torque remained at almost 0.0 N/cm and did not show any evidence of viscosity change during melt modification. FTIR was used to characterize the resulting materials after vacuum drying at 50° C. for over 72 hours (FIG. 2). FTIR spectra of GMA could not be differentiated from PLA oligomers, thus FTIR was unable to verify existence of unsaturated PLA-GMA oligomeric material. However, FTIR of PLA-TMI material clearly showed absence of —NCO groups and presence of secondary amine —NH— groups, indicating formation of unsaturated PLA oligomers.

### Example 3

PLA oligomer and modified PLA oligomeric materials were further vacuum dried and then characterized for thermal analysis, and the results are shown in FIG. 3. Neat PLA oligomer showed multiple DSC endothermic peaks during heating, but it was difficult to define origins of individual peaks. Typically, PLA polymer has a glass transition temperature of ˜58° C. and a melting point of 170° C. It is tended to believe that low Mw PLA oligomer may have a lower Tg at ˜10° C. as evidenced by a tiny heat capacity step up in DSC curves. Multi-endothertnic peaks at 50-120° C. may correspond to melting of different imperfect PLA oligomer crystals. Addition of bulky chain ends may have caused more imperfect crystals, resulting in additional melting peaks. TGA results show that most of the modified PLA oligomers degrade less significantly at high temperatures, indicating that end-capping PLA oligomers improved thermal stability. In other words, end capping the carboxylic acid end groups makes the oligomeric materials more thermally stable.

While the foregoing is directed to embodiments of the present invention, other and further embodiments of the invention may be devised without departing from the basic scope thereof and the scope thereof is determined by the claims that follow.

What is claimed is:

1. A process of modifying polylactic acid comprising:
providing a first polylactic acid, wherein the first polylactic acid comprises a carboxylic acid end group; and
unsaturating the first polylactic acid to form second polylactic acid.

2. The process of claim 1 further contacting the first polylactic acid with a dual functional compound.

3. The process of claim 1, wherein the first polylactic acid is represented by the formula: 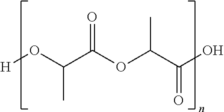
wherein n is a discrete number.

4. The process of claim 1, wherein the first polylactic acid is an oligomer.

5. The process of claim 1, wherein the first polylactic acid exhibits a number average molecular weight of from about 500 g/mol to about 200,000 g/mol.

6. The process of claim 1, wherein the first polylactic acid exhibits a number average molecular weight of from about 1000 g/mol to about 20,000 g/mol.

7. The process of claim 2, wherein the dual functional compound comprises a first functional group comprises a carbon-carbon double bond on one end and a second functional group capable of reacting with the carboxylic acid end group.

8. The process of claim 7, wherein the second functional group is selected from epoxy groups, isocyanate groups and combinations thereof.

9. The process of claim 2, wherein the dual functional compound is selected from glycidyl methacrylates, unsaturated isocyanates, epoxidized butadiene and combinations thereof.

10. The process of claim 2, wherein the contact comprises reactive extrusion.

11. A modified polylactic acid formed by the process of claim 1.

12. A process of forming polymeric compositions comprising:
providing an olefinic group; and
contacting the olefinic group with the modified polylactic acid of claim 11 under polymerization conditions to form a polymeric composition.

13. The process of claim 12, wherein the olefinic group is selected from styrene, acrylate, and combinations thereof.

14. The process of claim 12, wherein the olefinic group is an unsaturated polyolefin comprising polybutadiene.

15. A polymeric blend comprising:
a third polylactic acid;
an olefin based polymer; and
a polymeric composition formed by the process of claim 14, wherein the polymeric composition is adapted to compatibilize the third polylactic acid and olefin based polymer.

16. A co-extruded article comprising:
a first layer comprising a third polylactic acid;
a second layer comprising an olefin based polymer; and
a tie layer comprising a polymeric composition formed by the process of claim 13.

17. The process of claim 12, wherein the contact occurs in the presence of peroxide.

18. A process of forming polymeric compositions comprising polymerizing an unsaturated polylactic acid formed by:
contacting a first polylactic acid with a dual functional compound to form an unsaturated polylactic acid, wherein the first polylactic acid is represented by the formula: 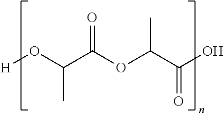
wherein n is a discrete number; and
polymerizing the unsaturated polylactic acid under polymerization conditions to form a polymeric composition.
